(19) United States
(12) Patent Application Publication
(10) Pub. No.: US 2025/0221855 A1
(43) Pub. Date: Jul. 10, 2025

(54) IMPLANTABLE SHUNTS WITH SELF-ADJUSTING PRESSURE RELIEF FEATURES AND ASSOCIATED SYSTEMS AND METHODS

(71) Applicant: Shifamed Holdings, LLC, Campbell, CA (US)

(72) Inventors: David Batten, San Francisco, CA (US); Signe Ruth Erickson, Redwood City, CA (US); Tessa Bronez, Campbell, CA (US)

(21) Appl. No.: 19/009,159

(22) Filed: Jan. 3, 2025

Related U.S. Application Data
(60) Provisional application No. 63/618,092, filed on Jan. 5, 2024.

(51) Int. Cl. A61F 9/007

(52) U.S. Cl. CPC A61F 9/00781; A61F 2250/0013; A61F 2250/0037

(57) ABSTRACT

The present technology is generally directed to implantable shunting systems including a shunting element and one or more self-adjusting pressure relief features disposed inside the shunting element. The shunting element can include inlets at a first end portion, outlets at a second end portion, and flow channels extending between corresponding inlets and outlets. The self-adjusting pressure relief features can be positioned within the flow channels. In some embodiments, the self-adjusting pressure relief features can switch between a closed state and an open state when the pressure differential across the pressure relief feature exceeds or falls below a predetermined pressure differential threshold. Different self-adjusting pressure relief features can be tuned to open and close at different thresholds. In some embodiments, the varying fluid resistances of the flow channels define different tiers, and the number of flow channels in each tier can be varied to provide a desired pressure-resistance relationship.

### CROSS-REFERENCE TO RELATED APPLICATION(S)

The present application claims the benefit of U.S. Provisional Patent Application No. 63/618,092, filed Jan. 5, 2024, the disclosure of which is incorporated herein by reference in its entirety.

### TECHNICAL FIELD

The present technology generally relates to implantable medical devices and, in particular, to implantable shunting systems with self-adjusting pressure relief features.

### BACKGROUND

Implantable shunts have been used to treat glaucoma by creating an artificial drainage pathway from the anterior chamber to relieve excess pressure in the anterior chamber. However, shunting too little or too much fluid from the anterior chamber via the shunt can result in problematic pressure levels in the eye. Therefore, there is a need for effective systems and methods for shunting fluid and adjusting pressure levels in the eye that are adjustable in response to changing pressure levels in the anterior chamber.

### BRIEF DESCRIPTION OF THE DRAWINGS

Many aspects of the present technology can be better understood with reference to the following drawings. The components in the drawings are not necessarily drawn to scale. Instead, emphasis is placed on illustrating clearly the principles of the present technology. Furthermore, components can be shown as transparent in certain views for clarity of illustration only and not to indicate that the component is necessarily transparent. Components may also be shown schematically.

FIG. 1A is a partially transparent, perspective view of a shunting system configured in accordance with an embodiment of the present technology.

FIG. 1B is an enlarged partially transparent, perspective view of a portion of the shunting system of FIG. 1A.

FIG. 2 is a schematic circuit diagram representative of a portion of the shunting system of FIG. 1A.

FIG. 3 is a schematic circuit diagram representative of another portion of the shunting system of FIG. 1A.

FIG. 4 is a flowchart illustrating a method for shunting fluid from a first region of a patient's eye to a second region of the patient's eye in accordance with an embodiment of the present technology.

### DETAILED DESCRIPTION

The present technology is generally directed to implantable shunting systems with self-regulating and/or self-adjusting pressure relief features, and associated systems and methods. Shunting systems are commonly used to promote the flow of fluid between a first body region, such as the anterior chamber of an eye, and a second body region, such as a surgically created bleb located in/on the eye (e.g., a subconjunctival bleb). However, it can be important to control the rate and timing of shunting fluid to maintain the fluid pressure level in the anterior chamber within a desired range. As described throughout this Detailed Description, it can be advantageous to selectively shunt fluid based on, for example, the pressure differential between the fluid pressure level at the first region and the fluid pressure level at the second region.

The implantable shunting systems configured in accordance with the present technology can include a shunting element and one or more self-regulating and/or self-adjusting pressure relief features disposed inside the shunting element. The shunting element can include inlets at a first end portion, outlets at a second end portion, and flow channels extending between corresponding inlets and outlets.

In some embodiments, the self-regulating pressure relief features are the flow channels themselves. For example, due to the relatively small dimensions (e.g., relatively small cross-sectional areas) of the channels, fluid may not begin to flow through a channel until a difference between the fluid pressure level at the first region and the fluid pressure level at the second region exceeds an inherent pressure differential threshold of the channel. Thus, when the pressure differential is less than the inherent pressure differential threshold, fluid does not flow through the channel. When the pressure differential is greater than the inherent pressure differential threshold, fluid flows through the channel. In such embodiments, the properties (e.g., dimensions) of individual channels and the total number of channels can determine the pressure-relief properties of the system.

In some embodiments, rather than relying upon the inherent pressure differential thresholds of the channels themselves, the systems described herein include self-regulating pressure relief features positioned within or proximate the flow channels. The pressure relief features can be configured to switch between a closed state and an open state when the pressure differential between the fluid pressure level at the first region and the fluid pressure level at the second region exceeds or falls below a predetermined pressure differential threshold. For example, in some embodiments the self-adjusting pressure relief features comprise pressure-sensitive valves. The valves can be tuned to open and close at desired pressure differential thresholds.

In some embodiments, the pressure differential thresholds can be set such that the pressure level at the anterior chamber of the patient's eye remains less than 5 mmHg, less than 10 mmHg, less than 15 mmHg, less than 20 mmHg, less than 25 mmHg, or other values. For example, in embodiments in which the channels themselves are the self-regulating pressure relief features, the threshold may be between about 1 mmHg and about 15 mmHg, or between about 2 mmHg and about 10 mmHg, or between about 3 mmHg and about 8 mmHg. In embodiments in which the channels include pressure-sensitive valves, the pressure-sensitive valves may be tuned to switch from the closed state and the open state when a pressure differential exceeds a predetermined threshold of between about 1 mmHg and about 20 mmHg, or between about 2 mmHg and about 15 mmHg, or between about 3 mm Hg and about 10 mmHg, or between about 4 mmHg and about 8 mmHg.

In some embodiments, the flow channels can vary in cross-sectional areas such that the fluid resistances therethrough differ between individual channels. For example, flow channels with relatively larger cross-sectional areas can have a lower fluid resistance than flow channels with relatively smaller cross-sectional areas. In embodiments with pressure-sensitive valves, the valves positioned in flow channels with different cross-sectional areas are tuned to open and close at different pressure differential thresholds. For example, valves positioned in flow channels with relatively larger cross-sectional areas (and thus relatively lower fluid resistances) can be set to open at a higher pressure differential threshold than valves positioned in flow channels with relatively smaller cross-sectional areas (and thus relatively higher fluid resistances).

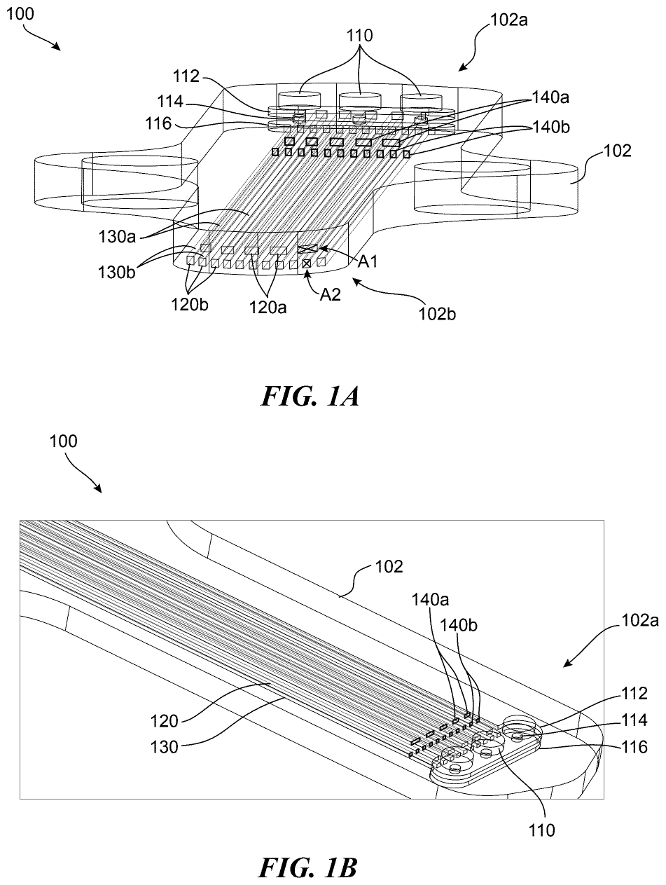

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the claims, but are not described in detail with respect to FIGS. 1A-4.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

As used herein, the use of relative terminology, such as “about,” “approximately,” “substantially” and the like refer to the stated value plus or minus ten percent. For example, the use of the term “about 100” refers to a range of from 90 to 110, inclusive. In instances in which the context requires otherwise and/or relative terminology is used in reference to something that does not include a numerical value, the terms are given their ordinary meaning to one skilled in the art.

FIG. 1A is a partially transparent, perspective view of a shunting system 100 (“the system 100”) configured in accordance with an embodiment of the present technology. The shunting system 100 can include a shunting element 102 with a first end portion 102a and a second end portion 102b opposite the first end portion 102a. The first end portion 102a can include a plurality of inlets 110, the second end portion 102b can include a first set of outlets 120a and a second set of outlets 120b (collectively referred to as “the outlets 120”), and the shunting element 102 can include a first subset or set of flow channels 130a and a second subset or set of flow channels 130b (collectively referred to as “the flow channels 130”). The first set of flow channels 130a can extend at least partially between the inlets 110 and the first set of outlets 120a, and the second set of flow channels 130b can extend at least partially between the inlets 110 and the second set of outlets 120b.

FIG. 1B is an enlarged partially transparent, perspective view of the first end portion 102a of the shunting system 100, rotated about 90 degrees clockwise relative to the orientation shown in FIG. 1A. Referring to FIGS. 1A and 1B together, the flow channels 130 can include a first set of flow channels 130a and a second set of flow channels 130b. As shown, the first set of flow channels 130a can extend between the first end portion 102a and the second end portion 102b at a different height than the second set of flow channels 130b (e.g., the first set of flow channels 130a extend in a different plane than the second set of flow channels 130b). The number of flow channels 130 in each of the first set of flow channels 130a and the second set of flow channels 130b can be one, two, three, four, five, six, seven, eight, nine, ten, eleven, twelve, or more. In some embodiments, the system 100 includes only the first set of flow channels 130a and the second set of flow channels 130b is omitted (e.g., all of the flow channels 130 exist in a common or substantially common plane). Regardless, individual channels of the first set of flow channels and individual channels of the second set of flow channels can each have a length of between about 5 mm and about 15 mm, or between about 6 mm and about 14 mm, or between about 7 mm and about 13 mm, or between about 8 mm and about 12 mm, such as about 8 mm, about 8.5 mm, about 9 mm, about 9.5 mm, about 10 mm, or about 10.5 mm. The foregoing dimensions are provided by way of example only—in other embodiments, individual channels of the first set of flow channels 130a and individual channels of the second set of flow channels 130b can have lengths outside of the foregoing ranges.

Each individual channel of the first set of flow channels 130a can have a first cross-sectional area A1 (as indicated in FIG. 1A at a representative outlet of the first set of outlets 120a) and each individual channel of the second set of flow channels 130b can have a second cross-sectional area A2 (as indicated in FIG. 1A at a representative outlet of the second set of outlets 120b). In the illustrated embodiment, the first cross-sectional area A1 is greater than the second cross-sectional area A2. In other embodiments, the first cross-sectional area A1 is less than or equal to the second cross-sectional area A2. In some embodiments, a dimension (e.g., a width, a height, a diameter, etc.) of individual channels of the first set of flow channels 130a and/or the second set of flow channels can be between about 25 μm and about 150 μm, or between about 30 μm and about 125 μm, or between about 35 μm and about 105 μm, or between about 40 μm and about 60 μm, or between about 45 μm and about 55 μm. The foregoing dimensions are provided by way of example only—in other embodiments, individual channels of the first set of flow channels 130a and individual channels of the second set of flow channels 130b can have dimensions outside of the foregoing ranges. In addition, although the flow channels 130 are illustrated as having a rectangular or even square cross-sectional shape, in other embodiments the flow channels 130 can have other cross-sectional shapes, including circular, oval-shaped, triangular, pentagonal, hexagonal, or other suitable shapes.

The shunting element 102 can also include a first reservoir 112 positioned fluidically between the inlets 110 and the first set of flow channels 130a, a second reservoir 116 positioned fluidically between the first reservoir 112 and the second set of flow channels 130b, and a plurality of channels 114 fluidically connecting the first reservoir 112 and the second reservoir 116. The first reservoir 112 and the second reservoir 116 can allow fluid to pool therein before flowing through the first set of flow channels 130a and the second set of flow channels 130b, respectively.

In some embodiments, each flow channel 130 can have an inherent pressure differential threshold that determines whether flow occurs through the channel 130 and is based at least in part on its resistance and/or dimensions (e.g., associated with the cross-sectional area of the channels 130). When the pressure differential between an inlet and outlet of an individual flow channel 130 is below the pressure differential threshold, in response, fluid does not flow through the individual flow channel 130. When the pressure differential between the inlet and the outlet of the individual flow channel 130 is above the pressure differential threshold, in response, fluid can flow through the individual flow channel 130. Representative inherent pressure differential thresholds can be between about 1 mmHg and about 15 mmHg, or between about 2 mmHg and 10 mmHg, or between about 3 mm Hg and about 8 mmHg, or between about 4 mmHg and 6 mmHg, including about 1 mmHg, about 2 mmHg, about 3 mmHg, about 4 mmHg, about 5 mmHg, about 6 mmHg, about 7 mmHg, about 8 mmHg, about 9 mmHg, about 10 mmHg, about 11 mmHg, about 12 mmHg, about 13 mmHg, about 14 mmHg, about 15 mmHg. The foregoing ranges and values are provided by way of example only—in other embodiments, the inherent pressure differential thresholds can be different than the foregoing ranges and values.

Individual flow channels 130 can have different pressure differential thresholds, e.g., based on their dimensions. For example, one or more of the first set of flow channels 130a can have a first channel fluid differential threshold, and one or more of the second set of flow channels 130b can have a second channel fluid differential threshold that is different than (e.g., greater than) the first channel fluid differential threshold. In general, flow channels 130 with smaller dimensions (e.g., smaller cross-sectional areas) will have a higher inherent pressure differential threshold.

In some embodiments, the shunting system 100 can be designed to passively regulate the fluid pressure level at the first region (e.g., at the anterior chamber of the eye) based on the inherent pressure differential threshold of each flow channel 130. For example, when the pressure differential across the system 100 exceeds the first channel fluid differential threshold, flow can occur through the first set of flow channels 130a. However, because the first channel fluid differential threshold is less than the second channel fluid differential threshold, flow may not occur through the second set of flow channels 130b. Instead, flow will only occur through the second set of flow channels 130b if the pressure differential across the system 100 also exceeds the second channel fluid differential threshold (e.g., if the first set of channels 130a did not provide adequate pressure relief). In this way, the system 100 can passively regulate the pressure differential across the system 100.

Moreover, the varying pressure differential thresholds can define one or more tiers of fluid resistances of the channels 130, and the number of channels 130 in each tier can be varied. For example, the shunting system 100 can include a greater number of channels 130 in the second set of flow channels 130b than in the first set of flow channels 130a. For example, in the illustrated embodiment, the shunting system 100 includes five channels 130 in the first set of flow channels 130a (e.g., low-resistance channels, first tier), and eleven channels 130 in the second set of flow channels 130b (e.g., high-resistance channels, second tier). In another example, the shunting system 100 can include one low-resistance channel (e.g., having the first cross-sectional area A1, first tier), two mid-resistance channels (e.g., each having a cross-sectional area between A1 and A2, second tier), and twenty high-resistance channels (e.g., each having the second cross-sectional area A2, third tier). One of ordinary skill in the art will appreciate that the shunting system 100 of the present technology can include one, two, three, four, five, six, or more tiers of channel resistance (e.g., low-resistance, mid-resistance, high-resistance), and can also include one, two, three, four, five, six, or more channels in each tier.

By including different numbers of channels 130 in the different tiers, the shunting system 100 of the present technology is expected to provide a desired pressure-resistance relationship for shunting fluid. Although the pressure differential threshold of each individual channel 130 remains the same, increasing the number of channels 130 within a tier can provide an effectively lower resistance for that tier compared to when including a single (or fewer) of the channels 130 within the tier. For example, if only a single high-resistance channel 130b is included, even if the fluid pressure differential exceeds the threshold of the single high-resistance channel 130b, the flow rate may not be sufficient for effectively shunting fluid. Thus, including a plurality of high-resistance channels 130b can allow the relatively low flow rate to add up across the multiple high-resistance channels 130b, providing an effectively lower resistance for the given tier. Accordingly, in illustrated embodiment, the effective combined resistance of the first tier defined by the first set of flow channels 130a may be higher than the effective combined resistance of the second tier defined by the second set of flow channels 130b, even though individual first channels 130a have a lower pressure differential threshold than individual second channels 130b. This is because there are more second channels 130b than first channels 130a. In some embodiments, the number of tiers and the number of channels 130 in each tier can be set such that the fluid pressure level at the first region (e.g., at the anterior chamber of the eye) remains below about 25 mmHg, below about 20 mmHg, below about 15 mmHg, below about 10 mmHg, below about 5 mmHg, or other pressure values.

In some embodiments, rather than relying solely on the inherent pressure differential thresholds of the channels 130, the shunting system 100 can also include one or more self-adjusting pressure relief features 140a, 140b (collectively referred to as “the features 140”), which are shown schematically in FIGS. 1A and 1B. The features 140 can be positioned in corresponding ones of the flow channels 130. In particular, the first set of flow channels 130a can be gated by a first set of features 140a, and the second set of flow channels 130b can be gated by a second set of features 140b. The features 140 can be configurable between an open state and a closed state. In the open state, the features 140 can allow fluid flow in the corresponding flow channel 130. In the closed state, the features 140 can prevent or substantially prevent fluid flow in the corresponding flow channel 130. In some embodiments, each feature 140 can be configured to automatically (e.g., passively) switch from the closed state to the open state when a pressure differential between either side of the feature 140 exceeds a corresponding predetermined pressure differential threshold. In some embodiments, each feature 140 can be configured to automatically (e.g., passively) switch from the open state to the closed state when the pressure differential between either side of the feature 140 falls below the corresponding predetermined pressure differential threshold. In some embodiments, each feature 140 can have a fixed predetermined pressure differential threshold. In some embodiments, each feature 140 can have a variable predetermined pressure differential threshold that can be tuned as desired.

The features 140 can have varying predetermined pressure differential thresholds such that different features 140 switch between the open state and the closed state at different pressure differentials. For example, in some embodiments each of the first set of features 140a can be configured to open at a first pressure differential, and each of the second set of features can be configured to open at a second pressure differential that is different than the first pressure differential. In other embodiments, individual ones of the first set of features 140a can be configured to open at different pressure differentials, and individual ones of the second set of features 140b can be configured to open at different pressure differentials. Representative pressure differential thresholds can be between about 1 mmHg and about 20 mmHg, or between about 2 mmHg and 15 mmHg, or between about 3 mm Hg and about 10 mmHg, or between about 4 mmHg and 8 mmHg, including about 1 mmHg, about 2 mmHg, about 3 mmHg, about 4 mmHg, about 5 mmHg, about 6 mmHg, about 7 mmHg, about 8 mmHg, about 9 mmHg, about 10 mmHg, about 11 mmHg, about 12 mmHg, about 13 mmHg, about 14 mmHg, about 15 mmHg, about 16 mmHg, about 17 mmHg, about 18 mmHg, about 19 mmHg, or about 20 mmHg. The foregoing ranges and values are provided by way of example only—in other embodiments, the features 140 can be tuned to operate at pressure differential thresholds different than the foregoing ranges and values.

In some embodiments, each of the features 140 comprises a pressure-sensitive valve or other type of valve. Representative types of valves include, but are not limited to, flap valves, membrane valves, ball valves, duckbill valves, needle valves, plug valves, butterfly valves, gate valves, check valves, pinch valve, globe valves, or other suitable valves. In some embodiments, the shunting system 100 does not include a shape memory actuator configured to control fluid flow through the flow channels 130.

Regardless of whether the system 100 includes the features 140 or relies upon the inherent pressure differential thresholds to regulate flow, the shunting system 100 can be sized and shaped to be implanted in a patient's eye to shunt fluid therefrom and treat medical conditions such as glaucoma. For example, the shunting element 102 can is sized and shaped such that, when implanted in the patient's eye, the first end portion 102a is positioned at least partially within a first region of the patient's eye (e.g., an anterior chamber of the eye) and the second end portion 102b is positioned at least partially within a second region of the patient's eye (e.g., a bleb surgically formed on the patient's eye).

In operation, the shunting system 100 can selectively shunt fluid from the anterior chamber to the bleb, thereby selectively altering (e.g., lowering) the fluid pressure level at the anterior chamber. More specifically, the fluid can enter the shunting element 102 via the inlets 110 and flow out of the first set of outlets 120a and the second set of outlets 120b via the first set of flow channels 130a and the second set of flow channels 130b, respectively, when the corresponding inherent pressure differential thresholds are exceeded, and/or when the corresponding features 140 are in the open state. When the inherent pressure differential thresholds are not exceeded, and/or when the corresponding features 140 are in the closed state, fluid cannot flow through those respective flow channels 130. As discussed above, the features 140 can switch between the open state and the closed state depending on whether the pressure differential at each feature 140 is above or below the predetermined pressure differential threshold. The pressure differential can be affected at least partially by the fluid pressure level at the first region. Thus, in some embodiments, the predetermined pressure differential threshold of each feature 140 can be set such that the fluid pressure level at the first region (e.g., at the anterior chamber of the eye) remains below about 25 mmHg, below about 20 mmHg, below about 15 mmHg, below about 10 mmHg, below about 5 mmHg, or other pressure values.

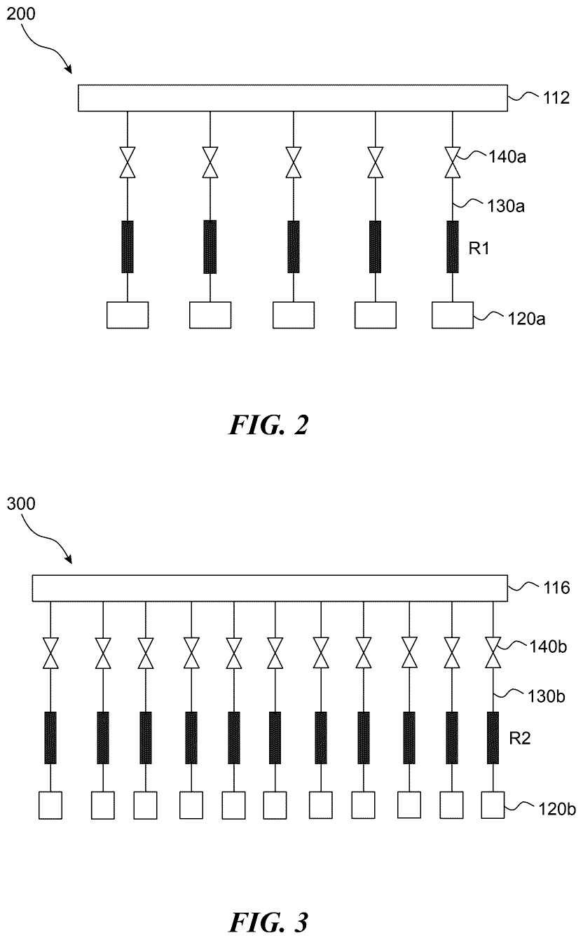

FIG. 2 is a schematic circuit diagram 200 (“the diagram 200”) that models a portion of the shunting system 100 shown in FIGS. 1A and 1B. More specifically, the diagram 200 schematically illustrates the first reservoir 112, the first set of outlets 120a, the first set of flow channels 130a extending therebetween, and the first set of features 140a positioned in corresponding ones of the first set of flow channels 130a. As discussed above with reference to FIGS. 1A and 1B, fluid from the first region (e.g., the anterior chamber of the eye) can enter the first reservoir 112 via the inlets 110 (not shown in FIG. 2) and flow through the first set of flow channels 130a depending on the state (e.g., open or closed) of the first set of features 140a. As also discussed above with reference to FIGS. 1A and 1B, each of the first set of flow channels 130a can have the first cross-sectional area A1, which can effectively translate into a first fluid resistance R1, schematically illustrated as a resistor in the diagram 200.

FIG. 3 is a schematic circuit diagram (“the diagram 300”) that models another portion of the shunting system 100 shown in FIGS. 1A and 1B. More specifically, the diagram 300 schematically illustrates the second reservoir 116, the second set of outlets 120b, the second set of flow channels 130b extending therebetween, and the second set of features 140b positioned in corresponding ones of the second set of flow channels 130b. As discussed above with reference to FIGS. 1A and 1B, fluid from the first region (e.g., the anterior chamber of the eye) can enter the second reservoir 112 via the inlets 110, the first reservoir 112, and the channels 114, and flow through the second set of flow channels 130b depending on the state (e.g., open or closed) of the second set of features 140b. As also discussed above with reference to FIGS. 1A and 1B, each of the second set of flow channels 130b can have the second cross-sectional area A2, which can effectively translate into a second fluid resistance R2, schematically illustrated as a resistor in the diagram 300.

Referring to FIGS. 1A-3 together, because the first cross-sectional area A1 of the first set of flow channels 130a is greater than the second cross-sectional area A2 of the second set of flow channels 130b, the first fluid resistance R1 can be smaller than the second fluid resistance R2. In some embodiments, the first set of features 140a and the second set of features 140b can be configured to open at different pressure differential thresholds to keep the overall pressure in the anterior chamber below about 20 mmHg, 15 mmHg, 10 mmHg, 5 mmHg, or other pressure values. For example, each of the first set of features 140a can have a first predetermined pressure differential threshold, and each of the second set of features 140b can have a second predetermined pressure differential threshold less than the first predetermined pressure differential threshold.

As described above, in some embodiments the shunting system 100 does not include the features 140 and instead relies upon the fluid resistances (e.g., R1, R2) of the channels 130 to regulate whether fluid flows through the corresponding channels 130. In some embodiments, the number of tiers of fluid resistances and the number of channels 130 in each tier can be set such that the fluid pressure level at the first region (e.g., at the anterior chamber of the eye) remains below about 25 mmHg, below about 20 mmHg, below about 15 mmHg, below about 10 mmHg, below about 5 mmHg, or other pressure values.

Those of ordinary skill in the art will appreciate that the shunting system 100 can have different configurations than the illustrated embodiment. For example, the shunting element 102 may include a different number of flow channels 130 and a corresponding number of features 140. The flow channels 130 may all have the same cross-sectional area. Alternatively, the flow channels 130 may have three or more different cross-sectional areas. The features 140 may all have the same predetermined pressure differential threshold. Alternatively, the features 140 may have three or more different pressure differential thresholds. Moreover, while the flow channels 130 in the illustrated embodiment extend linearly, the flow channels 130 can extend along different paths (e.g., curved, having cross-sectional areas that vary along the length of the flow channel 130) in other embodiments. Those of ordinary skill in the art will also appreciate that each feature 140 can be tuned independently based on the fluid resistance in the corresponding flow channel 130 (e.g., at least partially affected by the cross-sectional area of the flow channel), the fluid pressure levels at the anterior chamber and/or the bleb of the patient (which may differ across different patients), and/or other factors.

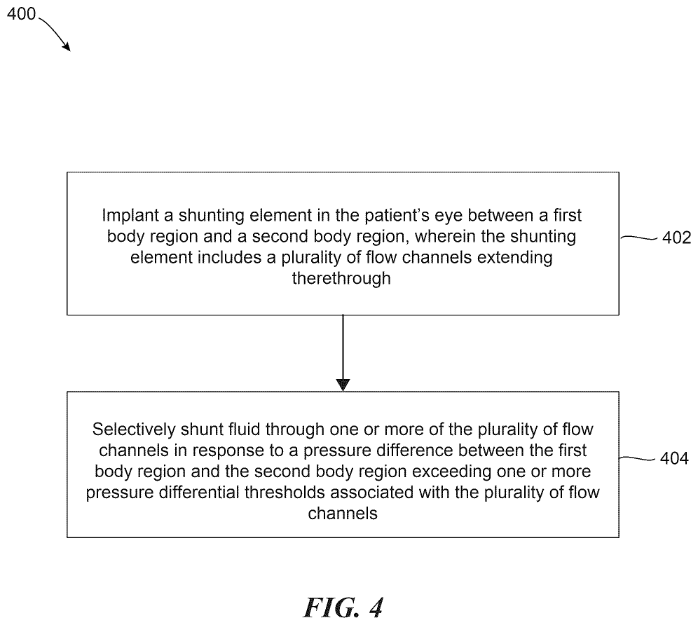

FIG. 4 is a flowchart illustrating a method 400 for shunting fluid from a first region of a patient's eye to a second region of the patient's eye, in accordance with an embodiment of the present technology. While the steps of the method 400 are described below in a particular order, one or more of the steps can be performed in a different order or omitted, and the method 400 can include additional and/or alternative steps. Additionally, although the method 400 may be described below with reference to the embodiments of the present technology described herein, the method 400 can be performed with other embodiments of the present technology.

The method 400 begins at block 402 by implanting a shunting element in the patient's eye such that a first end portion of the shunting element is positioned at least partially within the first region of the patient's eye and a second end portion of the shunting element opposite the first end portion is positioned at least partially within the second region of the patient's eye. The shunting element can include at least one inlet at the first end portion, a plurality of outlets at the second end portion, and a plurality of flow channels fluidly coupling the at least one inlet and the plurality of outlets.

At block 404, the method 400 continues by selectively shunting fluid through one or more of the plurality of flow channels in response to a pressure difference between the first region and the second region exceeding one or more pressure differential thresholds associated with the plurality of flow channels.

In some embodiments, selectively shunting fluid comprises (i) shunting fluid through a first subset of the plurality of flow channels in response to the pressure difference between the first region and the second region exceeding a first pressure differential threshold inherent to the first subset of the plurality of flow channels, and (ii) preventing fluid flow through a second subset of the plurality of flow channels in response to the pressure difference between the first region and the second region being less than a second pressure differential threshold inherent to the second subset of the plurality of flow channels.

In some embodiments, selectively shunting fluid comprises (i) switching first self-adjusting pressure relief features positioned in a first subset of the plurality of flow channels from a closed state to an open state in response to the pressure difference between the first region and the second region exceeding a first pressure differential threshold of the first self-adjusting pressure relief features, and (ii) maintaining second self-adjusting pressure relief features positioned in a second subset of the plurality of flow channels in the closed state response to the pressure difference between the first region and the second region being less than a second pressure differential threshold of the second self-adjusting pressure relief features.

### CONCLUSION

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. While steps are presented in a given order, alternative embodiments may perform steps in a different order. Moreover, the various embodiments described herein may also be combined to provide further embodiments. Reference herein to “one embodiment,” “an embodiment,” or similar formulations means that a particular feature, structure, operation, or characteristic described in connection with the embodiment can be included in at least one embodiment of the present technology. Thus, the appearances of such phrases or formulations herein are not necessarily all referring to the same embodiment.

For ease of reference, identical reference numbers are used to identify similar or analogous components or features throughout this disclosure, but the use of the same reference number does not imply that the features should be construed to be identical. Indeed, in many examples described herein, identically numbered features have a plurality of embodiments that are distinct in structure and/or function from each other. Furthermore, the same shading may be used to indicate materials in cross section that can be compositionally similar, but the use of the same shading does not imply that the materials should be construed to be identical unless specifically noted herein.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Where the context permits, singular or plural terms may also include the plural or singular term, respectively. Additionally, the term “comprising” is used throughout to mean including at least the recited feature(s) such that any greater number of the same feature and/or additional types of other features are not precluded. Directional terms, such as “upper,” “lower,” “front,” “back,” “vertical,” and “horizontal,” may be used herein to express and clarify the relationship between various elements. It should be understood that such terms do not denote absolute orientation. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A glaucoma shunting system for shunting fluid from a first region of a patient's eye to a second region of the patient's eye, the system comprising:
a shunting element including a first end portion and a second end portion opposite the first end portion, wherein the first end portion includes an inlet and the second end portion includes a plurality of outlets,
wherein the shunting element is sized and shaped such that, when the system is implanted in the patient's eye, the first end portion is positioned at least partially within the first region of the patient's eye and the second end portion is positioned at least partially within the second region of the patient's eye; and
a plurality of flow channels fluidly coupling the inlet with the plurality of outlets, wherein the plurality of flow channels includes one or more first flow channels and one or more second flow channels,
wherein the individual first flow channels have a first pressure differential threshold, and wherein, when the system is implanted in the patient's eye, fluid flows through each of the one or more first flow channels only when a pressure difference between the first region and the second region exceeds the first pressure differential threshold, and
wherein the individual second flow channels have a second pressure differential threshold greater than the first pressure differential threshold, and wherein, when the system is implanted in the patient's eye, fluid flows through each of the one or more second flow channels only when the pressure difference between the first region and the second region exceeds the second pressure differential threshold.

2. The system of claim 1 wherein the shunting element includes fewer first flow channels than second flow channels.

3. The system of claim 1 wherein the plurality of flow channels further comprises one or more third flow channels, and wherein the individual third flow channels each have a third pressure differential threshold greater than the first pressure differential threshold and less than the second pressure differential threshold, and further wherein the shunting element includes more third flow channels than first flow channels and fewer third flow channels than second flow channels.

4. The system of claim 1 wherein the first and second pressure differential thresholds are selected to maintain a pressure at the first region of the patient's eye below 15 mmHg.

5. The system of claim 1 wherein the first and second pressure differential thresholds are selected to maintain a pressure at the first region of the patient's eye below 10 mmHg.

6. The system of claim 1 wherein each of the plurality of flow channels has a cross-sectional dimension between 35-105 μm.

7. The system of claim 1 wherein each of the plurality of flow channels has a length between 6-14 mm.

8. The system of claim 1 wherein:
each of the one or more first flow channels has a first cross-sectional area, and wherein the first pressure differential threshold is based at least in part on the first cross-sectional area; and
each of the one or more second flow channels has a second cross-sectional area smaller than the first cross-sectional area, and wherein the second pressure differential threshold is based at least in part on the second cross-sectional area.

9. The system of claim 1 wherein the shunting element further comprises a reservoir fluidly coupling the at least one inlet and the plurality of flow channels, and wherein the reservoir is configured to allow fluid to pool therein before flowing through the plurality of flow channels.

10. The system of claim 1 wherein the one or more first flow channels extend along a first plane and the one or more second flow channels extend along a second plane, and wherein the shunting element further comprises:
a first reservoir fluidly coupling the at least one inlet and the one or more first flow channels, wherein the first reservoir is configured to allow fluid to pool therein before flowing through the one or more first flow channels; and
a second reservoir fluidly coupling the first reservoir and the one or more second flow channels, wherein the second reservoir is configured to allow fluid to pool therein before flowing through the one or more second flow channels.

11. The system of claim 1 wherein the shunting element does not include a shape memory actuator configured to control fluid flow through the plurality of flow channels.

12. The system of claim 1 wherein the shunting element does not include a self-adjusting pressure relief feature configured to control fluid flow through the plurality of flow channels.

13. The system of claim 1, further comprising a plurality of self-adjusting pressure relief features, each positioned in a corresponding one of the plurality of flow channels.

14. A glaucoma shunting system for shunting fluid from a first region of a patient's eye to a second region of the patient's eye, the glaucoma shunting system comprising.
a shunting element including a first end portion having at least one inlet, a second end portion opposite the first end portion and having a plurality of outlets, and a plurality of flow channels fluidly coupling the at least one inlet and the plurality of outlets, wherein the shunting element is sized and shaped such that, when the system is implanted in the patient's eye, the first end portion is positioned at least partially within the first region of the patient's eye and the second end portion is positioned at least partially within the second region of the patient's eye; and
a plurality of self-adjusting pressure relief features, wherein individual self-adjusting pressure relief features are positioned in corresponding individual flow channels and have corresponding pressure differential thresholds, and
wherein each self-adjusting pressure relief feature is transitionable between a closed state and an open state and is configured to:
allow fluid flow in the corresponding flow channel when in the open state,
prevent fluid flow in the corresponding flow channel when in the closed state, and
switch between the closed state and the open state when a pressure difference between a first fluid pressure level at the first region and a second fluid pressure level at the second region exceeds the corresponding pressure differential threshold.

15. The system of claim 14 wherein the plurality of self-adjusting pressure relief features comprises a plurality of pressure-sensitive valves.

16. The system of claim 14 wherein the corresponding pressure differential threshold of each of the self-adjusting pressure relief features is set such that the first fluid pressure level at the first region remains below 10 mmHg.

17. The system of claim 14 wherein:
the plurality of flow channels comprises:
a first flow channel with a first cross-sectional area, and
a second flow channel with a second cross-sectional area smaller than the first cross-sectional area, and
the plurality of self-adjusting pressure relief features comprises:
a first self-adjusting pressure relief feature positioned in the first flow channel and having a first pressure differential threshold, and
a second self-adjusting pressure relief feature positioned in the second flow channel and having a second pressure differential threshold less than the first pressure differential threshold.

18. A method for shunting fluid from a first region of a patient's eye to a second region of the patient's eye, the method comprising:
implanting a shunting element in the patient's eye such that a first end portion of the shunting element is positioned at least partially within the first region of the patient's eye and a second end portion of the shunting element opposite the first end portion is positioned at least partially within the second region of the patient's eye, wherein the shunting element includes at least one inlet at the first end portion, a plurality of outlets at the second end portion, and a plurality of flow channels fluidly coupling the at least one inlet and the plurality of outlets; and
selectively shunting fluid through one or more of the plurality of flow channels in response to a pressure difference between the first region and the second region exceeding one or more pressure differential thresholds associated with the plurality of flow channels.

19. The method of claim 18 wherein selectively shunting fluid comprises:
shunting fluid through a first subset of the plurality of flow channels in response to the pressure difference between the first region and the second region exceeding a first pressure differential threshold inherent to the first subset of the plurality of flow channels; and
preventing fluid flow through a second subset of the plurality of flow channels in response to the pressure difference between the first region and the second region being less than a second pressure differential threshold inherent to the second subset of the plurality of flow channels.

20. The method of claim 18 wherein selectively shunting fluid comprises:
switching first self-adjusting pressure relief features positioned in a first subset of the plurality of flow channels from a closed state to an open state in response to the pressure difference between the first region and the second region exceeding a first pressure differential threshold of the first self-adjusting pressure relief features; and
maintaining second self-adjusting pressure relief features positioned in a second subset of the plurality of flow channels in the closed state response to the pressure difference between the first region and the second region being less than a second pressure differential threshold of the second self-adjusting pressure relief features.
